(19) United States
(12) Patent Application Publication
(10) Pub. No.: US 2019/0201345 A1
(43) Pub. Date: Jul. 4, 2019

(54) EXCIPIENT AND TABLET

(71) Applicant: JAPAN ANTIVIRUS RES. INST., LTD., Nara-shi, Nara (JP)

(72) Inventor: Akihiro FUJINO, Nara-shi, Nara (JP)

(73) Assignee: JAPAN ANTIVIRUS RES. INST., LTD., Nara-shi, Nara (JP)

(21) Appl. No.: 16/314,219

(22) PCT Filed: Jun. 27, 2017
(86) PCT No.: PCT/JP2017/023475

§ 371 Date: Dec. 28, 2018

(30) Foreign Application Priority Data
Jun. 28, 2016 (JP) 2016-128150

(51) Int. Cl. A61K 9/20; A23L 29/212; A23L 29/262; C11D 3/22

(52) U.S. Cl. CPC A61K 9/2059; A23L 29/212; A23L 29/262; A61K 9/2054; A23P 10/28; C11D 3/221; C11D 3/222; A23V 2002/00; A61K 9/2018

(57) ABSTRACT

The present invention provides an excipient which can form a tablet, which is capable of rapidly disintegrating in water or in the body, from a functional powder such as a powdered cleaning agent or a powdered medicine. The excipient of the present invention is an excipient for tableting a powder and characterized by containing: starch, crystalline cellulose, and one or more hardening agents selected from the group consisting of maltitol, isomalt, maltose, and lactose. Thus, the excipient of the present invention facilitates tableting of a powder by a conventional procedure. The resulting tablet has excellent solubility in water, and dissolves rapidly after the tablet is placed in water. In addition, the resulting tablet has excellent hardness and can generally retain a predetermined shape with little damage, such as chipping, caused by external forces applied during storage or transportation.

### TECHNICAL FIELD

The present invention relates to an excipient and a tablet.

### BACKGROUND ART

Conventionally, there has been provided a cleaning tablet in which a cleaning agent is in the form of a tablet and which is used by dissolving in water at the time of use.

As such a cleaning tablet, for example, Patent Document 1 proposes a multi-phase cleaning tablet for use in a washing machine, wherein the tablet comprises: a) a first phase in the form of a shaped body having at least one mould therein; and b) a second phase in the form of a compressed body adhesively contained within said mould, wherein the tablet composition comprises one or more cleaning active ingredients which is predominantly concentrated in the second phase, and wherein the second phase additionally comprises a binder.

Also, in the field of medicines, powdered medicines are tableted because a patient is likely to spill such a powdered medicine when taking it, or in order to ensure that the patient takes a prescribed amount of the medicine specified by a prescription.

### CITATION LIST

### Patent Literature

Patent Literature 1: WO00/04115

### SUMMARY OF INVENTION

### Technical Problem

However, the multi-phase cleaning tablet of Patent Literature 1 has a problem that the tablet has insufficient solubility in water, so that it takes time to dissolve the tablet in water.

The present invention provides an excipient which can form a tablet, which has excellent hardness and is capable of rapidly disintegrating in water or in the body, from a powder such as a powdered cleaning agent or a powdered medicine, and a tablet which is prepared by tableting using the excipient.

### Solution to Problem

The excipient of the present invention is characterized by containing:

one or more hardening agents selected from the group consisting of maltitol, isomalt, maltose, and lactose,

starch, and

crystalline cellulose.

### Advantageous Effects of Invention

The excipient of the present invention facilitates tableting a powder by a conventional procedure. The resulting tablet has excellent solubility in water, and dissolves rapidly after the tablet is placed in water. In addition, the resulting tablet has excellent hardness and can generally retain a predetermined shape with little damage, such as chipping, caused by external forces applied during storage or transportation.

### DESCRIPTION OF EMBODIMENTS

The excipient of the present invention contains starch, crystalline cellulose, and one or more hardening agents selected from the group consisting of maltitol, isomalt, maltose, and lactose.

The excipient of the present invention is an excipient for tableting a powder, and contains starch, crystalline cellulose, and one or more hardening agents selected from the group consisting of maltitol, isomalt, maltose, and lactose.

The excipient contains starch. The starch is not particularly limited, and examples thereof include potato starch, sweet potato starch, corn starch, tapioca starch, wheat flour starch, rice starch, bean starch, kudzu starch, bracken starch, and katakuriko (potato starch). Among these, tapioca starch is preferable. The starch may be used alone or in combination of two or more kinds thereof.

The excipient contains crystalline cellulose as a binder. “Crystalline cellulose” means one obtained by depolymerizing partially a natural cellulosic material with an acid, and then purifying depolymerized products. Examples of the crystalline cellulose include metros, methylcellulose, carboxymethylcellulose, hydroxyethylcellulose, hydroxypropylmethylcellulose, hypromellose, and carmellose sodium. Examples of the natural cellulosic material include cellulose obtained from plants such as wood, bamboo, wheat straw, rice straw, cotton, ramie, bagasse, kenaf, and beet, cellulose obtained from sea squirt, and cellulose obtained from bacteria such as acetic acid bacteria. The natural cellulosic material may be used alone or in combination of two or more kinds thereof as a raw material. The crystalline cellulose may be used alone or in combination of two or more kinds thereof.

The average degree of polymerization of the crystalline cellulose is preferably 500 or less. The average degree of polymerization of the crystalline cellulose can be measured by the reduced specific viscosity method using a copper ethylenediamine solution as specified in the crystal cellulose confirmation test (3) of “the 14th revised Japanese Pharmacopoeia” (published by Hirokawa Shoten). The average degree of polymerization of the crystalline cellulose is preferably 10 to 500, more preferably 10 to 300. The crystalline cellulose having the average degree of polymerization falling within the above-mentioned range can provide the tablet obtained using the excipient with excellent disintegrating property in water or in the body.

As a method of controlling the average degree of polymerization of the crystalline cellulose, for example, hydrolysis treatment of a natural cellulosic material can be mentioned. By the hydrolysis treatment, depolymerization of the amorphous cellulose inside the natural cellulosic material proceeds, so that the average degree of polymerization becomes small. At the same time, since impurities such as hemicellulose and lignin are removed in addition to the above-mentioned amorphous cellulose by the hydrolysis treatment, crystalline cellulose in which the inside of the natural cellulosic material is made porous is obtained.

The method of hydrolysis is not particularly limited, and examples thereof include acid hydrolysis, thermal hydrolysis, steam explosion, and microwave decomposition.

The particle shape (L/D) of the crystalline cellulose is preferably 20 or less, more preferably 15 or less, further preferably 10 or less, still more preferably 5 or less, particularly preferably less than 5, and most preferably 4 or less. The lower limit of the particle shape (L/D) of the crystalline cellulose is 1 from its definition.

The particle shape (L/D) of the crystalline cellulose refers to the value measured by the following procedure. First, crystalline cellulose is prepared as a pure water suspension having a concentration of 1% by mass, and stirred for 5 minutes at a rotation speed of 15,000 rpm using a high shear homogenizer (for example, “Excel Auto Homogenizer ED-7” manufactured by Nippon Seiki Co., Ltd.) to produce an aqueous dispersion. The aqueous dispersion is diluted with pure water to make a 0.1 to 0.5 mass % dispersion liquid. The dispersion liquid is cast on mica and dried by air. Crystalline cellulose in the air-dried product is observed with a high-resolution scanning microscope (SEM) or atomic force microscope (AFM). One hundred pieces are arbitrarily extracted from the observed crystal cellulose, and the major axis (L) and the minor axis (D) of each crystal cellulose in the observed direction are measured to calculate the major axis (L)/minor axis (D). The arithmetic mean value of the major axis (L)/minor axis (D) of the 100 pieces of crystal cellulose is defined as the particle shape (L/D) of the crystal cellulose. The major axis (L) and the minor axis (D) of the crystalline cellulose are measured by the following procedure: A true circle of a minimum diameter capable of enclosing crystalline cellulose is drawn. Further, an ellipse of a minimum area capable of enclosing the crystalline cellulose is drawn. A major axis of the ellipse is conformed to a diameter of the true circle. The major axis and the minor axis of the ellipse are regarded as the major axis (L) and the minor axis (D) of the crystalline cellulose, respectively.

The content of the crystalline cellulose in the excipient is preferably 5 to 20 parts by mass, more preferably 6 to 18 parts by mass, further preferably 7 to 16 parts by mass, particularly preferably 8 to 15 parts by mass, and most preferably 8 to 12 parts by mass, per 100 parts by mass of starch. The crystalline cellulose contained in an amount of 5 parts by mass or more improves the hardness of the tablet obtained using the excipient. The crystalline cellulose contained in an amount of 20 parts by mass or less improves the disintegrating property of the tablet obtained using the excipient.

The excipient contains one or more materials selected from the group consisting of maltitol, isomalt, maltose, and lactose as a hardening agent. The hardening agent preferably includes isomalt. The hardening agent may be used alone or in combination of two or more kinds thereof.

When two or more kinds of the hardening agents are used in combination, the hardening agents preferably contain isomalt and one or more compounds selected from the group consisting of maltitol, maltose, and lactose.

The content of the hardening agent in the excipient is preferably 0.2 to 10 parts by mass, more preferably 0.3 to 8 parts by mass, further preferably 0.5 to 5 parts by mass, and particularly preferably 0.6 to 3 parts by mass, and most preferably 0.7 to 2 parts by mass, per 100 parts by mass of starch. The hardening agent contained in an amount of 0.2 parts by mass or more improves the hardness of the tablet obtained using the excipient. The hardening agent contained in an amount of 10 parts by mass or less improves the disintegrating property of the tablet obtained using the excipient.

The excipient may contain an additive such as a lubricant and an enzyme within a range that does not impair the physical properties of the excipient. Examples of the lubricant include calcium stearate, sodium stearate, potassium stearate, and magnesium stearate.

The method of producing the excipient is not particularly limited as long as the constituent components can be uniformly mixed, and for example, the excipient can be produced by uniformly mixing starch, crystalline cellulose, and a hardening agent using a general-purpose mixing apparatus.

The excipient is used to tablet a powder. The powder is not particularly limited and examples thereof include a powdered cleaning agent, a powdered medicine, a powdered seasoning, a powdered food, a powdered health food, and a powdered health supplementary hood (supplement).

The method of tableting a powder using the excipient may include mixing a powder serving as a raw material and the excipient, followed by tableting the mixture (tableting). Any known method, for example, a direct tableting method or a dry granule compression method, may be adopted as a method of tableting a powder using the excipient. Specific examples of such a known method include (1) a method of tableting after mixing a powder serving as a raw material and the excipient, and an additive as necessary (direct tableting method), and (2) a method of producing granules by mixing a powder serving as a raw material and the excipient, and an additive as necessary, and tableting the granules (dry granule compression method). Thus, use of the above-described excipient can facilitate tableting of various powders by a known method. In the tablet obtained by tableting a powder using the excipient, the ratio of the content of the powder serving as the raw material to the content of the excipient (the content of the powder/the content of the excipient) is preferably 6.4 or less. In the tablet obtained by tableting a powder using the excipient, the ratio of the content of the powder serving as a raw material to the content of the excipient (the content of the powder/the content of the excipient) is preferably 0.1 or more.

The tablet obtained using the above-described excipient has excellent disintegrating property with respect to water. Thus, when the powder is, for example, a cleaning agent, the tablet rapidly disintegrates by simply placing the tablet in, for example, a washing machine, and the cleaning agent can be dissolved in water, so that the cleaning effect of the cleaning agent can be exhibited satisfactorily.

When the powder is a medicine and a patient takes the tablet, the tablet rapidly disintegrates by body fluid in the body, and the medicine can be smoothly absorbed in the body to develop excellent medicinal effects.

### EXAMPLES

The present invention will be described more specifically by illustrating examples, but the invention is not limited by these examples.

### Examples 1 to 27, and Comparative Examples 1 to 9

Tapioca starch, corn starch, potato starch, and rice starch as starch; crystalline cellulose 1 (trade name “Ceolus” manufactured by Asahi Kasei Chemicals Corporation), crystalline cellulose 2 (trade name “Hevaten 101” manufactured by Eiken Corporation), and crystalline cellulose 3 (trade name “Hevaten 102” manufactured by Eiken Corporation) as crystalline cellulose; and isomalt, maltitol, maltose, and lactose as a hardening agent were supplied to a stirring machine in respective predetermined amounts shown in Tables 1 and 2 and mixed uniformly to produce respective excipients.

A tablet composition was prepared by uniformly mixing 60 parts by mass of a cleaning agent (trade name “Attack” manufactured by Kao Corporation) or a seasoning (trade name “Knorr Cup Soup” manufactured by Ajinomoto Co., Inc.) as a powder and 40 parts by mass of the excipient. The tablet composition was supplied to a tableting machine and tableted at a tableting pressure of 14 kN by a direct tableting method to obtain a tablet (thickness: 9 mm, weight: 3,500 mg).

The disintegrating property and hardness of the obtained tablets were measured by the following procedures, and the results are shown in Tables 1 and 2.

[Disintegrating Property]

The obtained tablet was placed in 1 liter of water at 24.9° C. After the tablet was placed in water in a static state, the water was stirred at 60 rpm from a time point when ⅔ volume of the tablet had disintegrated to a time point when the tablet had completely disintegrated. The time from placing the tablet in water in a static state to complete disintegration of the tablet was measured. The tablet was evaluated as “Bad” if it did not disintegrate even after 300 seconds had elapsed since it was placed in water in a static state.

[Hardness]

The hardness of the obtained tablet was measured using a hardness meter (trade name “Grain Rigidity Tester” manufactured by Fujiwara Scientific Company Co., Ltd.).

TABLE 1

| | Excipient (Parts By Mass) | | | | | | | | | | | | | |
|---|---|---|---|---|---|---|---|---|---|---|---|---|---|---|
| | | | | | Crystalline | | | | | | | | Disintegrating | |
| | Tapioca | Corn | Potato | Rice | Cellulose | | | Hardening Agent | | | | | Property | Hardness |
| | Starch | Starch | Starch | Starch | 1 | 2 | 3 | Isomalt | Maltitol | Maltose | Lactose | Powder | (Second) | (kgf) |
| Example 1 | 100 | 0 | 0 | 0 | 10 | 0 | 0 | 1.3 | 0 | 0 | 0 | Cleaning Agent | 66 | 6.0 |
| Example 2 | 100 | 0 | 0 | 0 | 6 | 0 | 0 | 1.3 | 0 | 0 | 0 | Cleaning Agent | 120 | 5.8 |
| Example 3 | 100 | 0 | 0 | 0 | 18 | 0 | 0 | 1.3 | 0 | 0 | 0 | Cleaning Agent | 48 | 4.4 |
| Example 4 | 100 | 0 | 0 | 0 | 10 | 0 | 0 | 0.3 | 0 | 0 | 0 | Cleaning Agent | 75 | 4.4 |
| Example 5 | 100 | 0 | 0 | 0 | 10 | 0 | 0 | 8 | 0 | 0 | 0 | Cleaning Agent | 121 | 4.8 |
| Example 6 | 100 | 0 | 0 | 0 | 10 | 0 | 0 | 0 | 0.3 | 0 | 0 | Cleaning Agent | 100 | 4.0 |
| Example 7 | 100 | 0 | 0 | 0 | 10 | 0 | 0 | 0 | 1.3 | 0 | 0 | Cleaning Agent | 112 | 4.1 |
| Example 8 | 100 | 0 | 0 | 0 | 10 | 0 | 0 | 0 | 8 | 0 | 0 | Cleaning Agent | 140 | 4.4 |
| Example 9 | 100 | 0 | 0 | 0 | 10 | 0 | 0 | 0 | 0 | 0.3 | 0 | Cleaning Agent | 80 | 4.1 |
| Example 10 | 100 | 0 | 0 | 0 | 10 | 0 | 0 | 0 | 0 | 1.3 | 0 | Cleaning Agent | 95 | 4.3 |
| Example 11 | 100 | 0 | 0 | 0 | 10 | 0 | 0 | 0 | 0 | 8 | 0 | Cleaning Agent | 130 | 4.5 |
| Example 12 | 100 | 0 | 0 | 0 | 10 | 0 | 0 | 0 | 0 | 0 | 0.3 | Cleaning Agent | 80 | 4.0 |
| Example 13 | 100 | 0 | 0 | 0 | 10 | 0 | 0 | 0 | 0 | 0 | 1.3 | Cleaning Agent | 95 | 4.2 |
| Example 14 | 100 | 0 | 0 | 0 | 10 | 0 | 0 | 0 | 0 | 0 | 8 | Cleaning Agent | 125 | 4.5 |
| Example 15 | 100 | 0 | 0 | 0 | 10 | 0 | 0 | 0.65 | 0.65 | 0 | 0 | Cleaning Agent | 67 | 4.9 |
| Example 16 | 100 | 0 | 0 | 0 | 10 | 0 | 0 | 0.65 | 0 | 0.65 | 0 | Cleaning Agent | 63 | 4.8 |
| Example 17 | 100 | 0 | 0 | 0 | 10 | 0 | 0 | 0.65 | 0 | 0 | 0.65 | Cleaning Agent | 59 | 4.7 |
| Example 18 | 100 | 0 | 0 | 0 | 10 | 0 | 0 | 0 | 0.65 | 0.65 | 0 | Cleaning Agent | 73 | 4.5 |

TABLE 2

| | Excipient (Parts By Mass) | | | | | | | | | | | | | |
|---|---|---|---|---|---|---|---|---|---|---|---|---|---|---|
| | | | | | Crystalline | | | | | | | | Disintegrating | |
| | Tapioca | Corn | Potato | Rice | Cellulose | | | Hardening Agent | | | | | Property | Hardness |
| | Starch | Starch | Starch | Starch | 1 | 2 | 3 | Isomalt | Maltitol | Maltose | Lactose | Powder | (Second) | (kgf) |
| Example 19 | 100 | 0 | 0 | 0 | 10 | 0 | 0 | 0 | 0.65 | 0 | 0.65 | Cleaning Agent | 78 | 4.6 |
| Example 20 | 100 | 0 | 0 | 0 | 10 | 0 | 0 | 0 | 0 | 0.65 | 0.65 | Cleaning Agent | 86 | 3.9 |
| Example 21 | 0 | 100 | 0 | 0 | 10 | 0 | 0 | 1.3 | 0 | 0 | 0 | Cleaning Agent | 140 | 4.8 |
| Example 22 | 0 | 0 | 100 | 0 | 10 | 0 | 0 | 1.3 | 0 | 0 | 0 | Cleaning Agent | 130 | 4.5 |
| Example 23 | 0 | 0 | 0 | 100 | 10 | 0 | 0 | 1.3 | 0 | 0 | 0 | Cleaning Agent | 135 | 4.5 |
| Example 25 | 100 | 0 | 0 | 0 | 0 | 10 | 0 | 1.3 | 0 | 0 | 0 | Cleaning Agent | 60 | 3.8 |
| Example 26 | 100 | 0 | 0 | 0 | 0 | 0 | 10 | 1.3 | 0 | 0 | 0 | Cleaning Agent | 58 | 4.0 |
| Example 27 | 100 | 0 | 0 | 0 | 10 | 0 | 0 | 1.3 | 0 | 0 | 0 | Seasoning | 66 | 4.2 |
| Comparative | 100 | 0 | 0 | 0 | 10 | 0 | 0 | 0 | 0 | 0 | 0 | Cleaning Agent | 102 | 2.1 |
| Example 1 | | | | | | | | | | | | | | |
| Comparative | 100 | 0 | 0 | 0 | 0 | 0 | 0 | 1.3 | 0 | 0 | 0 | Cleaning Agent | 73 | 1.8 |
| Example 2 | | | | | | | | | | | | | | |
| Comparative | 100 | 0 | 0 | 0 | 0 | 0 | 0 | 0 | 1.3 | 0 | 0 | Cleaning Agent | 79 | 2.2 |
| Example 3 | | | | | | | | | | | | | | |
| Comparative | 100 | 0 | 0 | 0 | 0 | 0 | 0 | 0 | 0 | 1.3 | 0 | Cleaning Agent | 72 | 2.8 |
| Example 4 | | | | | | | | | | | | | | |
| Comparative | 100 | 0 | 0 | 0 | 0 | 0 | 0 | 0 | 0 | 0 | 1.3 | Cleaning Agent | 100 | 2.5 |
| Example 5 | | | | | | | | | | | | | | |
| Comparative | 0 | 0 | 0 | 0 | 10 | 0 | 0 | 1.3 | 0 | 0 | 0 | Cleaning Agent | Bad | 14.2 |
| Example 6 | | | | | | | | | | | | | | |
| Comparative | 0 | 0 | 0 | 0 | 10 | 0 | 0 | 0 | 1.3 | 0 | 0 | Cleaning Agent | Bad | 14.7 |
| Example 7 | | | | | | | | | | | | | | |
| Comparative | 0 | 0 | 0 | 0 | 10 | 0 | 0 | 0 | 0 | 1.3 | 0 | Cleaning Agent | Bad | 13.9 |
| Example 8 | | | | | | | | | | | | | | |
| Comparative | 0 | 0 | 0 | 0 | 10 | 0 | 0 | 0 | 0 | 0 | 1.3 | Cleaning Agent | Bad | 16.8 |
| Example 9 | | | | | | | | | | | | | | |

### INDUSTRIAL APPLICABILITY

The excipient of the present invention facilitates tableting of powders used in various applications, such as cleaning agents, medicines, seasonings, foods, health foods, and health supplemental foods (supplements). The obtained tablet rapidly disintegrates in water or in the body and has an excellent hardness.

### CROSS-REFERENCE TO RELATED APPLICATION

The present application claims the priority under Japanese Patent Application No. 2016-128150 filed on Jun. 28, 2016, the disclosure of which is hereby incorporated in its entirety by reference.

1. An excipient comprising:
one or more hardening agents selected from the group consisting of maltitol, isomalt, maltose, and lactose,
starch, and
crystalline cellulose.

2. The excipient according to claim 1, comprising 100 parts by mass of the starch, 5 to 20 parts by mass of the crystalline cellulose, and 0.2 to 10 parts by mass of the hardening agent.

3. The excipient according to claim 1, wherein the starch is tapioca starch.

4. The excipient according to claim 1, wherein the excipient is used for tableting a powder.

5. A tablet comprising:
the excipient according to claim 1; and
a powder serving as a raw material.

6. The tablet according to claim 5, wherein a ratio of a content of the powder to a content of the excipient (the content of the powder/the content of the excipient) is 6.4 or less.
